(19) United States
(12) Patent Application Publication
(10) Pub. No.: US 2007/0218471 A1
(43) Pub. Date: Sep. 20, 2007

(54) METHOD AND DEVICE FOR DETECTING DNA USING SURFACE-TREATED NANOPORE

(75) Inventors: Young Rok Kim, Yongin-si (KR); Jun Hong Min, Yongin-si (KR); In Ho Lee, Yongin-si (KR); Ah gi Kim, Yongin-si (KR); Kui Hyun Kim, Daejeon-si (KR)

Correspondence Address: CANTOR COLBURN, LLP, 55 GRIFFIN ROAD SOUTH, BLOOMFIELD, CT, 06002, US

(73) Assignee: SAMSUNG ELECTRONICS CO., LTD, Suwon-si (KR)

(21) Appl. No.: 11/550,099

(22) Filed: Oct. 17, 2006

(30) Foreign Application Priority Data
Oct. 17, 2005 (KR) 10-2005-0097648

(51) Int. Cl. C12Q 1/68; C12M 3/00

(52) U.S. Cl. 435/6; 435/287.2; 977/924

(57) ABSTRACT

Disclosed herein is a method for detecting DNA using a nanopore including treating the surface of a nanopore formed in a solid substrate with a substance carrying positive charges; introducing a DNA-containing sample into the surface-treated nanopore; and detecting electrical signals generated during translocation of the sample through the nanopore. Also disclosed herein is device for detecting DNA using a nanopore including a solid substrate including a nanopore, treated with a substance which carries positive charges to change a surface property of the nanopore so that the nanopore surface carries positive charges; an electrode applying voltage to the nanopore of the solid substrate; and a measurement unit measuring an electrical signal generated during translocation of a DNA-containing sample through the nanopore.

This application claims priority to Korean Patent Application No. 10-2005-0097648, filed Oct. 17, 2005, and all the benefits accruing therefrom under 35 U.S.C. § 119, the contents of which in its entirety are herein incorporated by reference.

### BACKGROUND OF THE INVENTION

1. Field of the Invention

The present invention relates to a method and device for detecting nucleic acids using a nanopore. More particularly, the invention relates to a method and device for detecting DNA without special labeling by using a nanopore, which may detect DNA having a size of less than 1 kilo-base-pairs (“kbps”) using a nanopore having changed surface properties, without special labeling.

2. Description of the Prior Art

Various methods for detecting target biomolecules in samples have been developed. Among these methods, the method of using nanopores for detection is now being considered as a high-sensitivity DNA detection system that may be similar to the method of using bio-pores.

Various DNA detection systems that use nanopores are known in the art. For example, U.S. Pat. No. 6,015,714 (entitled “Characterization of individual polymer molecules based on monomer-interface interactions”) discloses a method for sequencing DNA by distinguishing bases of DNA using the highly sensitive signals of nanopores, the method including: providing a small pore each having two pools between which one DNA can be placed; loading a DNA biopolymer into one of the pools; and taking measurements as the biopolymer passes through the pore.

Also, U.S. Pat. No. 6,362,002 (entitled “Characterization of individual polymer molecules based on monomer-interface interactions”) discloses a method of distinguishing a single-stranded nucleic acid from a double-stranded nucleic acid by providing a nanopore allowing sequential passage of bases of the a single-stranded DNA. In this disclosure, a double-stranded nucleic acid passes through a nanopore at a rate slower than that of a single-stranded nucleic acid, because the double stranded nucleic acid may be separated into single-stranded nucleic acids during its passage through the nanopore.

Also, U.S. Patent Publication No. 2003/0104428 (entitled “Method for characterization of nucleic acid molecules”) discloses a method for characterizing a sample DNA using a nanopore. In this disclosure, the method includes determining a specific sequence using either a substance recognizing a specified local area in a protein or DNA and observing changes in the signal amplitude caused by other substances that are bound to the DNA, thus detecting the specific base sequence of the DNA.

U.S. Pat. No. 6,428,959 (entitled “Methods of determining the presence of double stranded nucleic acids in a sample”) discloses a method of distinguishing a single-stranded nucleic acid from a double-stranded nucleic acid. The method includes translocating nucleic acids in an aqueous sample through a nanopore having a diameter ranging from 3 to 6 nanometers (nm) and monitoring the current amplitude through the nanopore during said translocating process.

However, such prior-art DNA detection methods and systems that use nanopores raise problems, because when these methods are applied, the detection of DNA having a size less than 2000 base pairs (bps) becomes difficult. Such difficulty rises from the extremely high passage rate of DNA through the nanopore. Other problems include the complicated structure of the DNA detection system and the difficulty of maintaining an appropriate DNA detection condition, mainly maintaining the diameter of the nanopore at less than 10 nm and preferably less than 5 nm.

Although many efforts have been made to form nanopores with a diameter as small as that of bio-pores, various problems resulting from the difficulty of forming such nanopores have been raised.

### BRIEF SUMMARY OF THE INVENTION

In an exemplary embodiment, the surface of a nanopore is treated with a substance carrying positive (+) charges to change the surface property of the nanopore. Treatment of substance carrying positive (+) charges to the nanopore surface renders the surface to carry positive (+) charges. Such treatment increases the interaction between the nanopore and DNA passing through said nanopore, thus allowing detection of DNA even with a nanopore having a relatively large diameter.

An exemplary embodiment provides a method and device for detecting DNA using a nanopore having modified surface properties. The method and device disclosed in the present invention allows detection of DNA in a sample through electrical signals without special labeling using a nanopore with a diameter ranging from 10 nm to 50 nm by extending the duration time of DNA translocation through the nanopore.

An exemplary embodiment provides a method and device for detecting DNA in a sample using a nanopore with a diameter ranging between 10-50 nm and having modified surface properties. Such method and device allows relatively easy detection of DNA without special labeling, even when the sample may be a polymerase chain reaction (“PCR”) product, particularly double-stranded DNA having a size of less than 1 kbps.

An exemplary embodiment provides a DNA detection device, which uses a nanopore having a significantly large diameter of 10-50 nm. The nanopore may be manufactured through a relatively simple process.

An exemplary embodiment provides a DNA detection device that may be applied to a lab-on-a-chip.

An exemplary embodiment provides a method for detecting DNA using a surface-treated nanopore. The method includes treating the surface of a nanopore formed on a solid substrate with a substance carrying positive (+) charges; providing a DNA-containing sample into the surface-treated nanopore; and detecting an electrical signal generated while the DNA translocates through the nanopore.

### BRIEF DESCRIPTION OF THE DRAWINGS

The above and other objects, features and advantages of the present invention will become more apparent from the following detailed description with reference to the following detailed description and accompanying drawings.

FIG. 1 shows an exemplary embodiment of an electrostatic interaction between the negative (−) charges in a DNA and the positive (+) charges on a nanopore surface according to the present invention;

FIGS. 2A and 2B schematically show the operational principle of an exemplary embodiment of a nanopore detection device according to the present invention;

FIGS. 3A and 3B show the electric currents measured in Example 1 and Comparative Example 1, respectively;

FIGS. 4A and 4B show the electric currents measured in Example 2 and Comparative Example 2, respectively;

FIG. 5 is a histogram showing the DNA translocation duration time, measured by applying a voltage of 500 millivolt (mV) to the devices in Examples 1 and 2, wherein the resulting signal data was measured for 2 minutes and the measured signal data was collected accordingly; and

FIG. 6 is a histogram showing the results of current blockade, measured by applying a voltage of 500 mV to the devices in Examples 1 and 2, wherein the resulting signal data was measured for 2 minutes and the measured signal data was collected accordingly.

### DETAILED DESCRIPTION OF THE INVENTION

The invention now will be described more fully hereinafter with reference to the accompanying drawings and examples, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art.

The terminology used herein is for the purpose of describing particular embodiments only and is not intended to be limiting of the invention. The terms “a” and “an” do not denote a limitation of quantity, but rather denote the presence of at least one of the referenced item. The term “or” means “and/or”. The terms “comprising”, “having”, “including”, and “containing” are to be construed as open-ended terms (i.e., meaning “including, but not limited to”).

Throughout the specification, the claims and the abstract, the term “nanopore” refers to a structure having a channel or pore having a nanometer diameter.

In a nanopore, an energy barrier, which needs to be overcome in order to translocate DNA contained in a sample through a nanopore, exists. . The height of the energy barrier is determined by various factors, such as electrostatic interactions or geometrical restrictions in the nanopore.

As illustrated in an exemplary embodiment of the nanopore detection device according to the present invention, the energy barrier of the nanopore may be increased by charging the surface of the nanopore so that the nanopore surface may carry positive (+) charges.

As illustrated in an exemplary embodiment in FIG. 1, DNA 100 contained in a sample carries negative (−) charges and the nanopore surface carries positive (+) charges. Thus, when DNA 100 in the sample translocates through the nanopore, the interaction between the nanopore surface having positive (+) charges and the DNA 100 having negative (−) charges will significantly increase. Accordingly, the translocation duration time, i.e., the time taken for the DNA 100 to translocate through the nanopore will increase.

An exemplary embodiment according to the present invention provides a method of changing the surface property of the nanopore, and accordingly increasing the DNA translocation duration time through the nanopore in a relatively simple manner. An exemplary embodiment of the method and device using nanopore for DNA detection according to the present invention maintains and does not reduce the signal amplitude, which occurs when using prior-art methods. In an exemplary embodiment, a relatively simple process may be used to prepare the nanopore having a diameter of 10-50 nm. In an exemplary embodiment of the method of forming a nanopore according to the present invention, the nanopore having a diameter of 10-50 nm can be formed by preparing a solid substrate 400, forming a 100 nm-diameter pore in the solid substrate 400 by using a focused ion beam (“FIB”) machine and reducing the 100 nm-diameter nanopore to a 10-50 nm nanopore by using an atomic layer deposition (“ALD”).

In an exemplary embodiment of the method according to the present invention, the nanopore having a diameter of 10-50 nm can be formed by depositing a silicon nitride membrane on a silicon substrate 400 using a low-pressure chemical vapor deposition (“LPCVD”), forming a 100 nm-diameter pore on the central portion of the silicon nitride membrane 400 using an FIB machine and depositing a thin layer made of aluminum oxide (Al₂O₃) 600 into the pore using ALD so as to adjust the diameter of the pore 200 to a size of 10-50 nm.

Exemplary embodiments of the method of forming a nanopore according to the present invention can be applied to nanopores having a diameter greater than 10 nm and less than 50 nm. Also, the upper limit of the nanopore 200 may be set to 50 nm, because when the diameter of the nanopore is larger than 50 nm, a detected signal cannot be accurately measured. In one embodiment, the nanopore has a diameter of about 30 nm.

The one or more substances having positive (+) charges 150, which may be used in exemplary embodiments according to the present invention to change the surface property of the nanopore, are selected from the group consisting of aminosilane, including aminopropyltriethoxysilane (APTES), nylon, nitrocellulose, spermidine and polylysine. As the surface property of the nanopore carrying positive (+) charges varies depending on the type and concentration of the positive-charge carrying substance that is treated on the nanopore surface, the detectable size of DNA 100 may be determined depending on the extent of change in the nanopore surface property.

In an exemplary embodiment, an electrical signal, may be measured to detect the DNA 100 in a sample using a nanopore. The electrical signal may be the amplitude of a current flowing through the nanopore. Since the translocation duration time of the sample DNA 100 through the nanopore will be increased due to the interaction between the positive (+) charges on the surface of the surface-treated nanopore and the negative (−) charges in the DNA 100 sample current blockade may be induced. Accordingly, DNA 100 in the sample may be detected by the current blockade signal. In other words, DNA 100 in the sample may be electrically detected by measuring the current blockade and the blockade time.

In an exemplary embodiment, sample containing DNA 100, which translocates through the nanopore, may be prepared in a liquid state by dissolving the sample in an electrically conductive solvent. Any of a number of electrically conductive solvent can be used as the solvent. In exemplary embodiments, the solvent may be an aqueous solvent, such as pure water or water containing at least one additive, such as buffer or salt. In an exemplary embodiment, potassium chloride (KCl) may be added to the aqueous solvent as an additive. In one exemplary embodiment, the solvent may be an ionized buffer solution, such as 1M KCl or 10 Mm Tris-hydrochloride (Tris HCl). The pH of the liquid sample may typically be about 6.0-9.0.

In exemplary embodiments, DNA 100 contained in the sample may be a PCR product, such as double-stranded DNA having a size of less than 1 kbps. In an exemplary embodiment, the size of the double-stranded DNA may range between 200 bps and 1000 bps. Thus, , the resulting products of a PCR can be analyzed in a relatively simple and fast manner.

An exemplary embodiment according to the present invention provides a device for detecting DNA using a nanopore. The device includes a solid substrate 400 having a nanopore, which may be treated with a substance carrying positive (+) charges to change the property of the nanopore surface so as to carry positive (+) charges on the surface, an electrode for applying voltage to the nanopore of the solid substrate 400 and a measurement unit for measuring an electrical signal generated during translocation of a DNA 100-containing sample through the nanopore.

The nanopore is a portion of a nanopore detection device. In the nanopore detection device, the nanopore surface may be treated with a substance having positive (+) charges and thus the required size of the nanopore 200 may be within the range of about 10-50 nm.

In an exemplary embodiment of a nanopore detection device, an electric field may be applied through the nanopore and the changes in the current through the nanopore may be monitored. Accordingly, based on the changes in the current in the nanopore, the target substance in the liquid sample may be detected while it translocates through the nanopore. The current amplitude through the nanopore may be monitored during the translocation of the target substance through the nanopore. The changes in the current amplitude value relate to the translocation of the target substance through the nanopore 200. Thus, the target substance can be effectively detected from the changes in the current amplitude value.

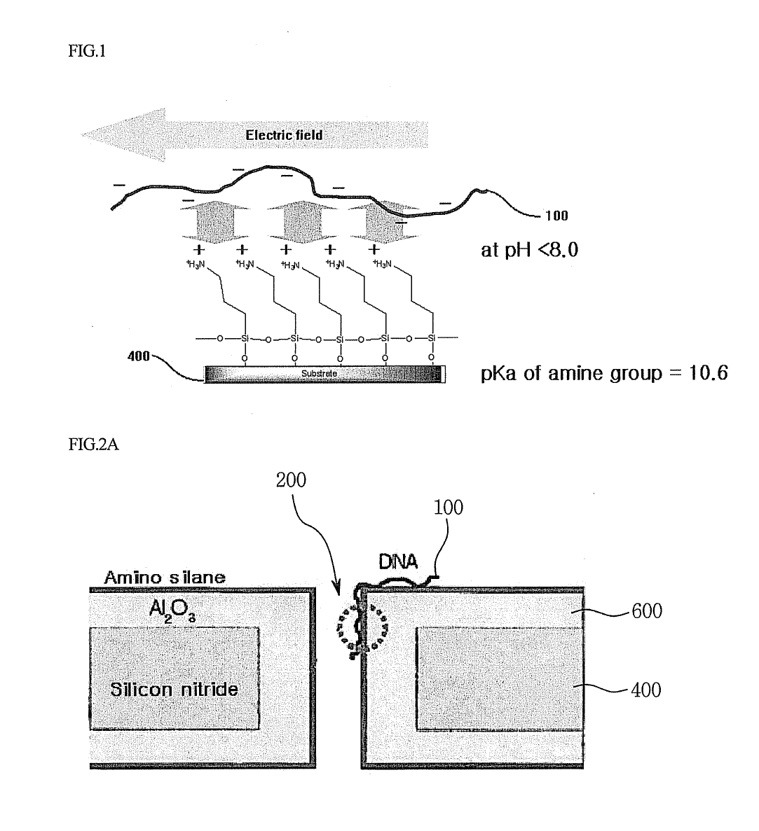

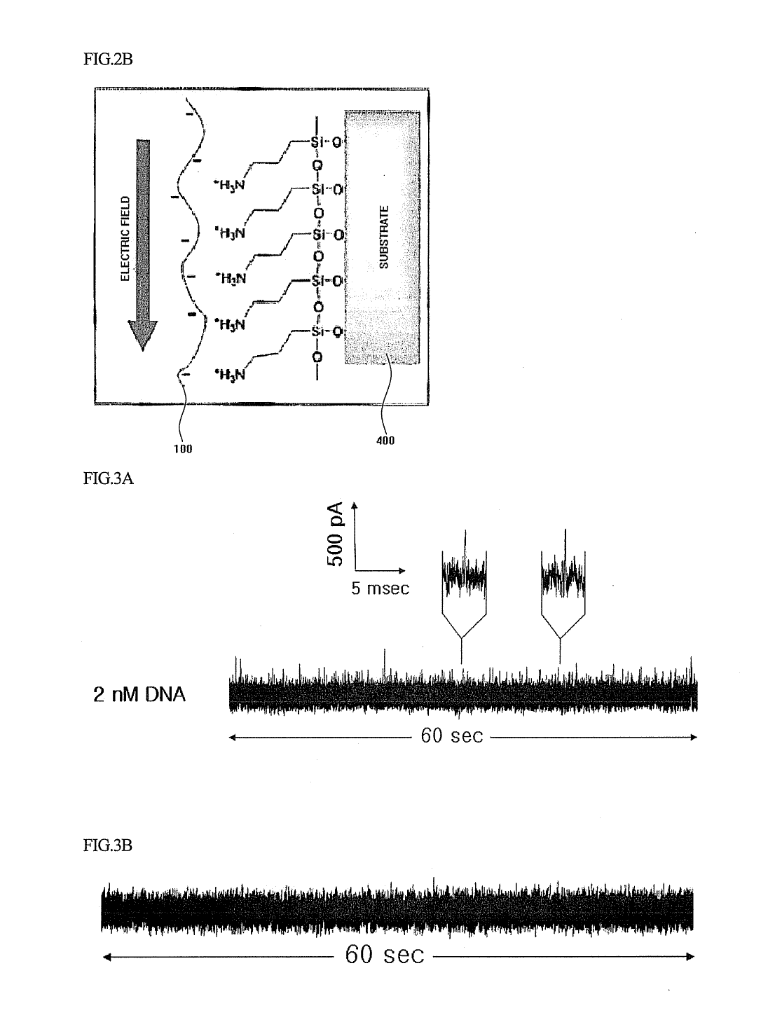

FIGS. 2A and 2B schematically shows the operational principle of the an exemplary embodiment of the nanopore detection device according to the present invention. The DNA 100 in a sample translocates through the nanopore 200, which has surface properties changed so that it carries positive (+) charges on its surface and electrostatically interacts with the nanopore 200 surface. Accordingly, the translocation time of the DNA 100 through the nanopore 200 may significantly increase compared to when the electrostatical interaction does not occur. Thus, even when the size of the DNA 100 is less than 1 kbps, a signal caused by the DNA 100 in the sample while the DNA 100 translocates through the nanopore 200 may be detected.

An exemplary embodiment of the nanopore 200 device according to the present invention may include a sample storage chamber (not shown) that is connected with the solid substrate 400. The sample, which is introduced into the nanopore 200, may be stored in the storage chamber. The sample may be a liquid substance containing a PCR product, i.e., DNA amplified using a PCR process with a size of less than 1 kbps.

In an exemplary embodiment, although the sample storage chamber may be constructed such that it stores a sample supplied from the external environment, the sample storage chamber may be constructed such that the desired sample may be produced using a DNA amplification unit (not shown). Such DNA application unit may include, but is not limited to, a PCR chip.

In an exemplary embodiment of the sample storage chamber, the sample storage chamber may be constructed such that it is connected with the DNA amplification unit through a fine channel having a nanometer-diameter channel, such that it can be supplied with the DNA-containing 100 sample.

In another embodiment of the nanopore detection device according to the present invention, the sample storage chamber may be connected with the DNA amplification unit by a process-on-a-chip or a lab-on-a-chip using microfluidic units and a micro electromechanical systems(“MEMS”) device.

Hereinafter, the present invention will be described in further detail with reference to the following examples. However, the following examples are for illustrative purposes only and are not to be construed to limit the scope of the present invention.

### Example 1

1. Formation of Solid Substrate having 30 nm Diameter Nanopore

A 250 nm silicon nitride was deposited on silicon substrate using low pressure chemical-vapor deposition (“LPCVD”). A thin free standing membrane of silicon nitride having a size of 30μm× 30 μm (Length×Width) was fabricated by opening a window in the unpolished side of the substrate using photo lithography, followed by reactive ion etching and KOH wet etching to remove the silicon. Then, a 100 nm diameter pore was formed in the central portion of the membrane using a FIB machine (focused ion beam machine) SMI2050 (manufactured by Seiko instrument Inc.). and a thin layer made of aluminum oxide (Al₂0₃) was deposited into the pore using atomic layer deposition so that the diameter of the pore was reduced to a size of 30 nm. The thickness of the deposited A1₂O₃ layer was measured with an ellipsometer. As a result, a cylindrical nanopore having a diameter of 30 nm was formed through the 320 nm thick membrane.

2. Surface Treatment of Nanopore

The solid substrate having the 30 nm-diameter nanopore was washed with piranha solution for 10 minutes and subsequently rinsed completely with distilled water. The washed substrate was immersed in an ethanol solution containing 1% (v/v) aminopropyl triethoxysilane (99%, Sigma-Aldrich) at room temperature for 1 hour. The substrate was completely rinsed by shaking it in the ethanol solution. Then, the substrate was dried with nitrogen gas. Then, the substrate was placed in an incubator at 100° C. for 2 hours. Within 24 hours after this treatment, the solid substrate was used.

3. Sample Preparation

Double-stranded DNAs each having a size of 539 bps and 910 bps , respectively, were prepared. Said DNAs were prepared by PCR amplifying a portion of a MODY3 gene. The PCR products were then purified using a QIAquick gel extraction kit (Qiagen).

4. Detection of DNA in sample 2 nM of the prepared 539 bps-double-stranded DNA was loaded together with an ionized buffer solution (1M KC1, 10 Mm Tris-HC1, pH 6.0) into a nanopore detection device having the surface-treated nanopore prepared as described above and an Ag/AgCl electrode for applying voltage through the nanopore. Then, a voltage of 500 mV was applied to the device and the resulting electrical signal was measured for 1 minute. The measurement results are shown in FIG. 3A.

### Comparative Example 1

Comparative example 1 was performed in the same manner as in Example 1, except that the surface treatment of the nanopore was not performed. The measurement results are shown in FIG. 3B.

### Example 2

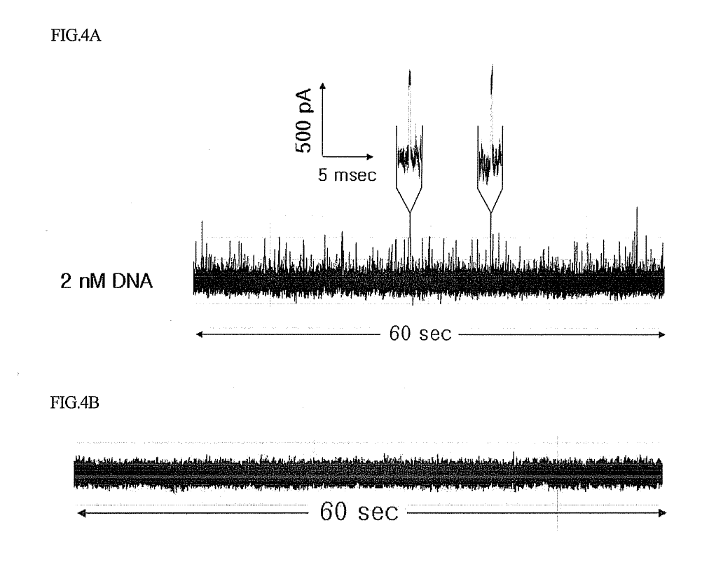

### Example 2 was performed in the same manner as in Example 1, except that the double-stranded DNA loaded into the inventive device had a size of 910 bps. The measurement results are shown in FIG. 4A.

### Comparative Example 2

Comparative example 2 was performed in the same manner as in Example 1, except that the surface treatment of the nanopore was not performed. The measurement results are shown in FIG. 4B.

As shown in FIGS. 3A, 3B, 4A and 4B, when the surface of the nanopore having a diameter of 30 nm was treated with the substance carrying positive (+) charges as disclosed in the exemplary embodiments according to the present invention, the changes in the electrical signal thereof were the same as the case where the sample did not include any DNA.

Thus, when the surface of the nanopore is treated with a substance carrying positive (+) charges as , DNA having a size of less than 1 kb can be detected in a relatively easy manner even with a nanopore having a diameter of 30 nm.

### Test Example

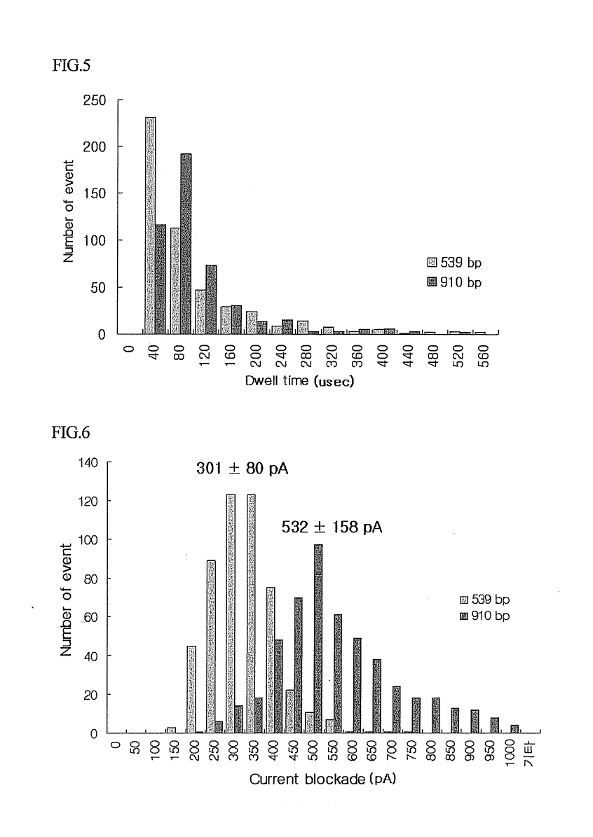

In order to examine the effect of DNA size on a measured signal, a voltage of 500 mV was applied to the devices of Examples 1 and 2 and the obtained signal data were collected for 2 minutes to determine the translocation duration time and current blockade through the nanopore. The results of the translocation duration time are shown in histograms in FIG. 5, and the results of the current blockade are shown as histograms in FIG. 6.

As illustrated in FIG. 5, the results of the translocation duration time of the short double-stranded DNAs were 56.7±2.6 microseconds (μsec) for the 539 bps DNA, and 106.7±28.6μsec for the 910 bps DNA.

Also, as illustrated in FIG. 6, the results of the average current blockade were 301±80 pA (at translocation number n=500) for the 539 bps DNA, and 532±158 pA (at translocation number n=500) for the 910 bps DNA.

These results show that as the size of double-stranded DNA increases, the duration time of DNA translocation through the surface-treated nanopore and the current blockade may be increased.

Although the exact reasons for such results are not known, it may be speculated that the folding of the DNA itself caused by an increase in the size of double-stranded DNA may be one of the reasons.

As described above, the use of the nanopore that was surface-treated with the substance carrying positive (+) charges shown in the exemplary embodiments of the method and device for detecting DNA using the nanopore according to the present invention enables detection of DNA having a size of less than 1 kbps. Thus, the such method and device shown in the exemplary embodiments according to the present invention can be conveniently applied to a DNA sensor in the form of a lab-on-a-chip, which allows relatively fast detection without labeling.

Exemplary embodiments of the method and device for detecting DNA using the nanopore according to the present invention have the following advantages.

In an exemplary embodiment, DNA in a sample may be detected by measuring an electrical signal caused by increasing the duration time of DNA translocation through the nanopore. The increased duration time of DNA translocation may be caused by the change in the surface property of the nanopore. Thus, even when the sample is a PCR product, particularly double-stranded DNA having a size of less than 1 kbps, the DNA can be detected using a nanopore having a diameter of 10-50 nm without special labeling, such that PCR results can be analyzed in a relatively simple and fast manner. Also, because the nanopore may be prepared by using a simple and convenient process the nanopore may be applied to a DNA detection device, which can be applied to a lab-on-a-chip.

Although the preferred embodiment of the present invention has been described for illustrative purposes, those skilled in the art will appreciate that various modifications, additions and substitutions are possible, without departing from the scope and spirit of the invention as disclosed in the accompanying claims.

What is claimed is:

1. A method for detecting DNA using a nanopore, the method comprising
treating the surface of a nanopore formed in a solid substrate with a substance carrying positive charges;
introducing a DNA-containing sample into the surface-treated nanopore; and
detecting electrical signals generated during translocation of the sample through the nanopore.

2. The method of claim 1, wherein the substance which carries positive charges is one or more selected from the group consisting of amino silane, nylon, nitrocellulose, spermidine and polylysine.

3. The method of claim 1, wherein the electrical signals are current blockade and blockade time.

4. The method of claim 1, wherein the nanopore has a size of about 10-50 nm.

5. The method of claim 1, wherein the DNA is a double-stranded DNA having a size of less than 1 kbps.

6. A device for detecting DNA using a nanopore, the device comprising:
a solid substrate, including a nanopore, treated with a substance which carries positive charges to change a surface property of the nanopore so that the nanopore surface carries positive charges;
an electrode applying voltage to the nanopore of the solid substrate; and
a measurement unit measuring an electrical signal generated during translocation of a DNA-containing sample through the nanopore.

7. The device of claim 6, further comprising a sample storage chamber, wherein the sample storage chamber is connected with the solid substrate and stores the sample introduced into the nanopore.

8. The device of claim 7, wherein the sample storage chamber comprises a DNA amplification unit or is connected with the DNA amplification unit.

9. The device of claim 6, wherein the substance which carries positive charges is one or more selected from the group consisting of amino silane, nylon, nitrocellulose, spermidine and polylysine.

10. The device of claim 6, wherein the nanopore has a size of about 10-50 nm.

11. The device of claim 6, wherein the DNA is a double-stranded DNA having a size of less than 1 kbps.
